(19) United States
(12) Patent Application Publication
(10) Pub. No.: US 2025/0028159 A1
(43) Pub. Date: Jan. 23, 2025

(54) MULTIMODAL MICROSCOPY SYSTEM

(71) Applicant: AGENCY FOR SCIENCE, TECHNOLOGY AND RESEARCH, Singapore (SG)

(72) Inventors: Shuyan ZHENG, Singapore (SG); Malini OLIVO, Singapore (SG)

(73) Assignee: AGENCY FOR SCIENCE, TECHNOLOGY AND RESEARCH, Singapore (SG)

(21) Appl. No.: 18/712,553

(22) PCT Filed: Nov. 23, 2022
(86) PCT No.: PCT/SG2022/050855

§ 371 Date: May 22, 2024

(30) Foreign Application Priority Data
Nov. 26, 2021 (SG) 10202113156S

(51) Int. Cl. G02B 21/12; A61B 1/04; A61B 1/07; G01N 21/64; G02B 21/16; G02B 21/26; G02B 21/36; G02B 27/14; G02B 27/30

(52) U.S. Cl. CPC G02B 21/12; A61B 1/07; G01N 21/6458; G02B 21/16; G02B 21/26; G02B 21/361; G02B 27/30; A61B 1/04; G02B 27/141

(57) ABSTRACT

Embodiments of the invention provide for a multimodal microscopy system. The multimodal microscopy system may include an illumination module configured to provide a light beam to a sample under test via a delivery path; and an optical arrangement configured to receive the light beam from the sample through a detection path to generate a microscopy image of the sample. The optical arrangement may be operable to switch among a plurality of imaging modalities using the detection path shared by the plurality of imaging modalities. The plurality of imaging modalities may include brightfield microscopy, fluorescent microscopy, and endoscopic microscopy. The multimodal microscopy system may be a multimodal hyperspectral microscopy system.

### RELATED APPLICATIONS

The present application claims priority to and the benefit of Singapore Patent Application 10202113156S, filed 26 Nov. 2021, which is hereby incorporated by reference in its entirety.

### TECHNICAL FIELD

Various embodiments relate to a microscopy system, and more particularly, a multimodal microscopy system.

### BACKGROUND

Most commonly used optical microscopes display images with only three wavelength bands in the visible spectrum, namely, red, green, and blue (RGB). However, with limitations in resolution, magnification, and surface view, conventional optical microscopes cannot provide sufficient information about a sample in the corresponding image display, especially for applications requiring detailed data. Therefore, spectral imaging technology, especially hyperspectral imaging technology which captures spectral information in the visible range as well as extends the wavelength range from visible to ultraviolet and infrared was developed and widely applied in various industries, such as astronomy, agriculture, food, military surveillance, biomedical research, medical diagnostics, and treatments.

Hyperspectral imaging microscopy technology is a combined technology of spectroscopy and digital imaging, that collects and processes information from across the electromagnetic spectrum or from the use of optical filters to capture a specific spectral range of wavelength bands for each pixel in an image.

A hyperspectral microscopy system uses transmitted, reflected, or re-emitted light captured from each point in the sample with spectroscopic methods such as visible, near-infrared, and fluorescence imaging to acquire three-dimensional data with two spatial dimensions and one spectral dimension of a single pixel to produce hyperspectral images. Certain samples leave a unique spectral signature in the electromagnetic spectrum, that enables the identification of the materials to make up a scanned sample. With at least the abovementioned advantages, the hyperspectral microscopy system becomes a powerful imaging tool for providing information about tissue physiology, morphology, and composition to biomedical research, medical diagnosis, and image guided-surgeries.

Moreover, for different types of samples, to better collect spatial and spectral information, various imaging modalities may be adopted such as brightfield microscopy, fluorescent microscopy, and endomicroscopy. For certain applications, two or more imaging modalities may be involved within the setting of a single examination. However, currently in existing systems, there is no single microscopy system that can accommodate more than two modalities, more specifically, brightfield, fluorescence, and endoscopic imaging modalities, without significant modifications to the setup for measurement.

A key challenge in integrating three imaging modalities into one single system is to minimize the footprint of the system while maintaining its high image resolutions. For example, an endoscopic imaging modality with external illumination conventionally requires its illumination probe (e.g. a light guide) to be placed separately from and at an angle to a collection probe, thereby enlarging the overall setup dimensions taken up by the whole system and thus its footprint.

Thus, there is a need for a single multimodal microscopy system that may be operable among three imaging modalities, namely brightfield, fluorescent, and endoscopic microscopy to perform RGB, monochrome, and/or hyperspectral imaging.

### SUMMARY OF INVENTION

According to an embodiment, a multimodal microscopy system is provided. The multimodal microscopy imaging system may include an illumination module configured to provide a light beam to a sample under test via a delivery path, an optical arrangement configured to receive the light beam from the sample through a detection path to generate a microscopy image of the sample. The optical arrangement may be operable to switch among a plurality of imaging modalities using the detection path shared by the plurality of imaging modalities, and the plurality of imaging modalities may include brightfield microscopy, fluorescent microscopy, and endoscopic microscopy.

### BRIEF DESCRIPTION OF DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1(a) shows a schematic diagram illustrating a multimodal microscopy system, according to various embodiments.

FIG. 1(b) shows a schematic diagram illustrating an optical arrangement of the multimodal microscopy system of FIG. 1(a), according to various embodiments.

FIG. 1(c) shows a schematic diagram illustrating an illumination module of the multimodal microscopy system of FIG. 1(a), according to various embodiments.

FIG. 2(a) shows a perspective view of a multimodal microscopy system configured for brightfield hyperspectral microscopy set up in a transmission mode using a broadband light source, according to various embodiments.

FIG. 2(b) shows a side partial view of the multimodal microscopy system of FIG. 2(a) when in operation with a light path, according to various embodiments.

FIG. 2(c) shows a perspective view of a multimodal microscopy system configured for brightfield hyperspectral microscopy set up in a reflection mode using a broadband light source, according to various embodiments.

FIG. 2(d) shows a side partial view of the multimodal microscopy system of FIG. 2(c) when in operation with a light path, according to various embodiments.

FIG. 3(a) shows a hyperspectral image of ex-vivo normal human liver cells taken by the multimodal microscopy system configured for brightfield hyperspectral microscopy as described in FIG. 2(a), using 32 spectral bands within the range of 420 nm to 730 nm with a spectral step of 10 nm.

FIGS. 3(b) to 3(e) are charts illustrating the spectral information of selected pixels representing different material compositions in the image of FIG. 3(a).

FIG. 4(a) shows a hyperspectral image of an ex-vivo cancerous human liver cell taken by the multimodal microscopy system configured for brightfield hyperspectral microscopy as described in FIG. 2(a), using 32 spectral bands within the range of 420 nm to 730 nm with a spectral step of 10 nm.

FIGS. 4(b) to 4(e) are charts illustrating the spectral information of selected pixels representing different material compositions in the image of FIG. 4(a).

FIG. 5(a) shows a graph illustrating a spectral reference library consisting of the transmittance spectra of normal liver nuclei and cancerous liver nuclei at selected points.

FIGS. 5(b) and 5(c) show hyperspectral images of normal liver cells and cancerous liver cells, respectively, taken by the multimodal microscopy system configured for brightfield hyperspectral microscopy as described in FIG. 2(a), using 32 spectral bands within the range of 420 nm to 730 nm with a spectral step of 10 nm, where each selected nuclei position is marked with a cross.

FIG. 6(a) shows a perspective view of a multimodal microscopy system configured for fluorescence hyperspectral microscopy set up in a transmission mode using either a narrowband light source or a broadband light source with an excitation filter, according to various embodiments.

FIG. 6(b) shows a side partial view of the multimodal microscopy system of FIG. 7(a) when in operation with a light path, according to various embodiments.

FIG. 6(c) shows a perspective view of a multimodal microscopy system configured for a fluorescence hyperspectral microscopy set up in a reflection mode using either a narrowband light source or a broadband light source with an excitation filter, according to various embodiments.

FIG. 6(d) shows a side partial view of the multimodal microscopy system of FIG. 6(c) when in operation with a light path, according to various embodiments.

FIGS. 7(a) to 7(f) are true-color hyperspectral images, under 20× magnification, of the six individual quantum dots at corresponding peak emission wavelengths of each quantum dot, taken by the fluorescent hyperspectral microscopy as described in FIG. 6(a).

FIG. 7(g) shows emission spectral profiles of quantum dots of hyperspectral images of FIGS. 7(a) to 7(f), according to various embodiments.

FIG. 8(a) is a true-color hyperspectral image, under 4× magnification, of the center of a mixture of six quantum dots, taken by the fluorescent hyperspectral microscopy as described in FIG. 6(a), according to various embodiments.

FIGS. 8(b) to 8(d) are charts of spectra of selected individual pixels in the image of FIG. 8(a), according to various embodiments.

FIG. 9(a) shows a side partial view of the multimodal microscopy system configured for endoscopic hyperspectral microscopy set up in a transmission mode with external illumination, according to various embodiments.

FIG. 9(b) shows a side partial view of the multimodal microscopy system configured for endoscopic hyperspectral microscopy set up in a reflection mode with external illumination, according to various embodiments.

FIG. 10(a) shows a side partial view of a fiber probe integrated with a metasurface at the facet thereof used for the multimodal microscopy system configured for endoscopic hyperspectral microscopy set up in a reflection mode with internal illumination, according to various embodiments.

FIG. 10(b) shows a geometric representation of the metasurface of FIG. 10(a), according to various embodiments.

FIG. 10(c) shows a side view of the multimodal microscopy system configured for endoscopic hyperspectral microscopy set up in a reflection mode with internal illumination, according to various embodiments.

FIG. 11(a) shows a partial perspective view of a connection between a fiber probe and a microscope body via a three-axis stage in part, according to one example.

FIGS. 11(b) and 11(c) respectively show the top and bottom perspective view of the connections between the fiber probe and an imaging plane of the microscope objective lens, as expanded from parts of FIG. 11(a).

FIG. 12(a) shows a graph illustrating the spectral reference library consisting of transmittance spectra of normal lung tissue and cancerous lung tissue averaged at selected points.

FIGS. 12(b) and 12(c) show reconstructed true-colour (RGB) images from the hyperspectral datacubes of normal lung tissue and cancerous lung tissue, respectively, taken by the multimodal microscopy system configured for endoscopic hyperspectral microscopy as described in FIG. 9(a), according to various embodiments.

FIGS. 13(a) and 13(b) show reconstructed true-colour (RGB) images of normal lung tissue and cancerous lung tissue, respectively, as re-iterated from FIGS. 12(b) and 12(c).

FIGS. 13(c) and 13(d) are the false-color representations of results of classified normal lung tissue and cancerous lung tissue, respectively by applying spectral analysis using a reference library.

FIG. 14(a) shows a graphical representation of simulated 3D light beam propagations of two excitation beams (off-axis focusing) and one collection beam (on-axis focusing) modelled based on fiber points with metasurface integration, according to one example.

FIG. 14(b) shows a perspective view of the overlapping region of the simulated three light beams of FIG. 14(a).

FIG. 15 shows a representative diagram illustrating an exemplary multimodal microscopy system (in parts) switchable among different imaging modalities of brightfield hyperspectral microscopy, fluorescence hyperspectral microscopy, and endoscopic hyperspectral microscopy, according to various embodiments.

### DETAILED DESCRIPTION

The following detailed description refers to the accompanying drawings that show, by way of illustrations, specific details, and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more embodiments to form new embodiments.

Embodiments described in the context of one of the systems are analogously valid for the other systems.

Features that are described in the context of an embodiment may correspondingly apply to the same or similar features in the other embodiments, even if not explicitly described in other embodiments. Furthermore, additions and/or combinations and/or alternatives described for a feature in the context of an embodiment may correspondingly be applicable to the same or similar feature in the other embodiments.

In the context of various embodiments, the articles “a”, “an”, and “the” as used with regard to a feature or element include a reference to one or more of the features or elements.

In the context of various embodiments, the terms “compact” and “integrated” may be used interchangeably.

As described herein, the expression “configured to” may be referred to as “constructed to”. The expression “system” may mean apparatus.

Various embodiments may provide a multimodal microscopy system for performing hyperspectral imaging. The multimodal microscopy system may also be used for non-hyperspectral imaging. When performing hyperspectral imaging, the system may achieve high spectral and spatial resolution in each imaging modality and may reduce the footprint of the entire system by sharing certain components of the system among the three imaging modalities as well as utilizing metasurface designs at the endoscope fiber facet. In particular, embodiments of the invention may provide for a multimodal microscopy system which includes an illumination module configured to provide a light beam to a sample, and an optical arrangement configured to receive the light beam from the sample, wherein the light beam travels along a delivery path, to the sample, and to a detection path, and wherein the detection path may be shared by a plurality of imaging modalities of the multimodal microscopy system. For example, the multimodal microscopy system may advantageously provide high spectral and spatial resolution hyperspectral images of the sample, and the multimodal arrangement which includes three imaging modalities may allow the sample to be studied by different imaging modalities within a compact arrangement. In addition, the compact arrangement may reduce the footprint of the system as compared to having separate systems for each different modality.

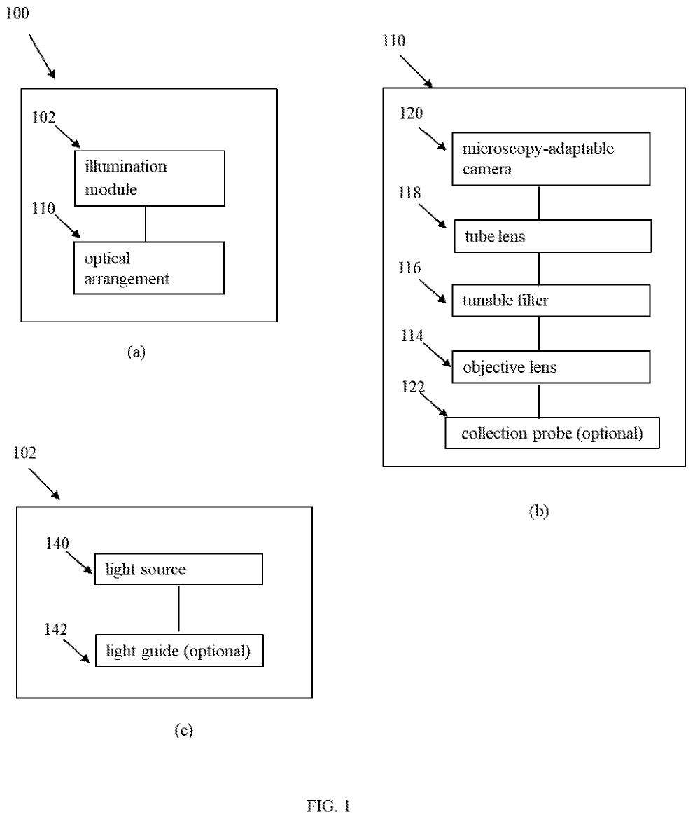

FIG. 1(a) shows a schematic diagram illustrating a multimodal microscopy system 100, in accordance with various embodiments.

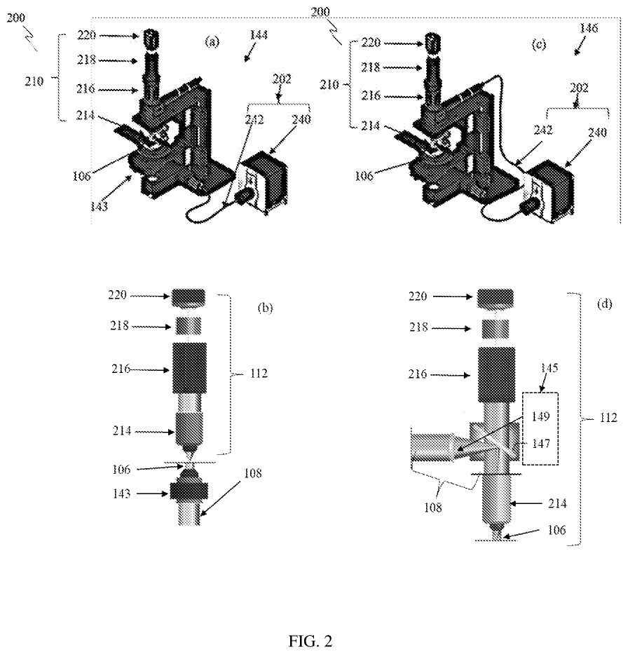

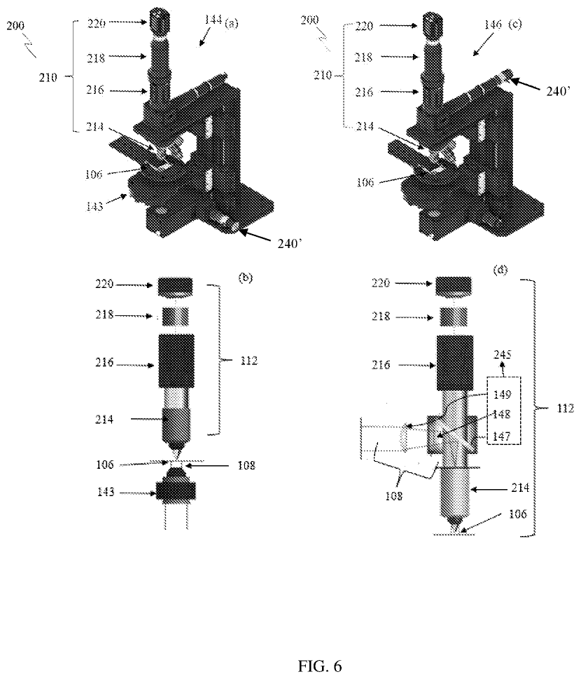

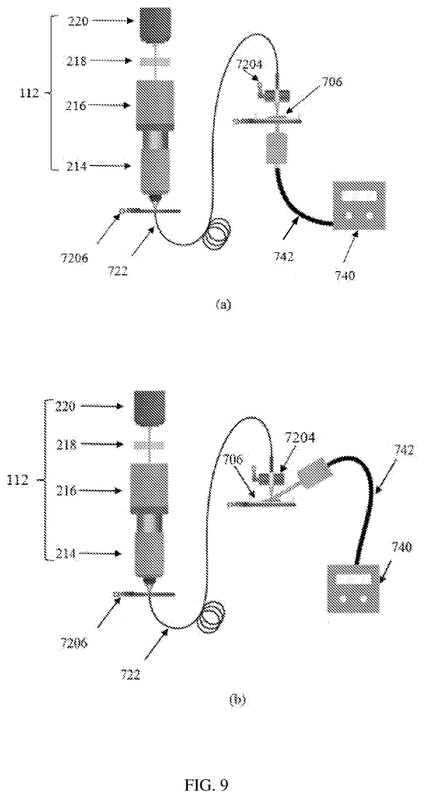

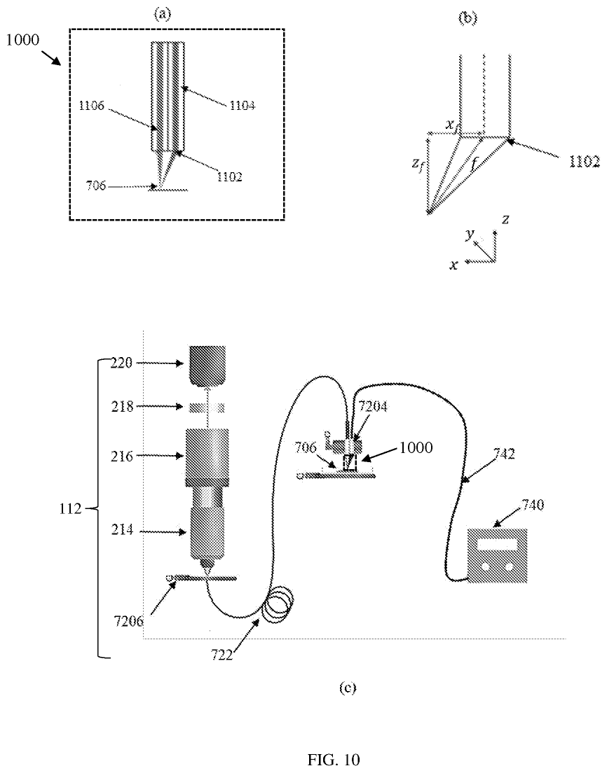

As seen in FIG. 1(a), the multimodal microscopy system 100 may include an illumination module 102 and an optical arrangement 110. The illumination module 102 may be configured to provide a light beam to a sample (e.g. 106 in FIGS. 2(a) to 2(d), 6(a) to 6(d), and 706 in FIGS. 9(a), 9(b), 10(a) and 10(c)) under test via a delivery path 108 (see e.g. FIGS. 2(b), 2(d), 6(b), and 6(d)) and the optical arrangement 110 may be configured to receive the light beam from the sample through a detection path (e.g. 112 in FIGS. 2(b), 2(d), 6(b), 6(d), 9(a), 9(b), and 10(c)) to generate a microscopy image of the sample. The optical arrangement 110 may be operable to switch among a plurality of imaging modalities by using the detection path shared by the plurality of imaging modalities. The plurality of imaging modalities may include brightfield microscopy, fluorescent microscopy, and endoscopic microscopy.

It should be appreciated that the multimodal microscopy system 100 may perform hyperspectral imaging, and in doing so, the plurality of imaging modalities may include brightfield hyperspectral microscopy, fluorescent hyperspectral microscopy, and endoscopic hyperspectral microscopy. The multimodal microscopy system 100 may also be used to perform non-hyperspectral imaging, without the need to change or significantly modify the components of the system 100. For simplicity, the description hereinbelow may relate to hyperspectral imaging, but it should be understood that the same may be applicable to non-hyperspectral imaging.

In other words, the multimodal microscopy system 100 may be a multimodal hyperspectral microscopy system. The brightfield microscopy, fluorescent microscopy, and endoscopic microscopy may be brightfield hyperspectral microscopy, fluorescent hyperspectral microscopy, and endoscopic hyperspectral microscopy, respectively. FIG. 15 shows a representative diagram illustrating an exemplary multimodal microscopy system, e.g. 100, (in parts) switchable among different imaging modalities of brightfield hyperspectral microscopy 1502, fluorescence hyperspectral microscopy 1504, and endoscopic hyperspectral microscopy 1506. Details on the brightfield hyperspectral microscopy 1502, the fluorescence hyperspectral microscopy 1504, and the endoscopic hyperspectral microscopy 1506 will be provided in the corresponding descriptions below for FIGS. 2(d), 6(d) and 10(c), respectively.

Making reference to FIGS. 1(a) to 1(c) and parts of FIGS. 2(a) to 2(d), 6(a) to 6(d), and 9(a) to 9(b), the illumination module 102 and the optical arrangement 110 may be optically coupled to carry the light beam and form a light path. The light beam may be emitted from the illumination module 102, transmitted to or reflected by the sample 106 to arrive through the detection path 112 and generate a microscopy image of the sample 106. The three hyperspectral imaging modalities, namely, brightfield hyperspectral microscopy, fluorescent hyperspectral microscopy, and endoscopic hyperspectral microscopy may be configured based on different illumination modules 102, but share the same optical arrangement 110 and detection path 112, thereby allowing the three hyperspectral imaging modalities to be configurable and compacted into one (single) multimodal microscopy system 100.

FIG. 1(b) shows a schematic diagram illustrating the optical arrangement 110, according to various embodiments.

As seen in FIG. 1(b), the optical arrangement 110 may include an objective lens 114 configured to receive the light beam from the sample 106, a tunable filter 116 optically coupled to the objective lens 114, a tube lens 118 optically coupled to the tunable filter 116, and a microscopy-adaptable camera 120 optically coupled to the tube lens 118. This configuration may be adopted when the plurality of imaging modalities may be switched to brightfield hyperspectral microscopy or fluorescent hyperspectral microscopy. The tunable filter 116 may be operable to select a specific wavelength and/or intensities or a specific range of wavelengths and/or intensities of the light beam. The microscopy-adaptable camera 120 may be for capturing the microscopy image of the sample (e.g. 106).

In a different embodiment, the optical arrangement 110 may also optionally include a collection probe 122 configured to receive the light beam from the sample 106. This configuration may be adopted when the plurality of imaging modalities may be switched to endoscopic hyperspectral microscopy. The collection probe 122 may be coupled to the objective lens 114 and configured to direct the light beam to the objective lens 114 and then to the tunable filter 116 optically coupled to the objective lens 114, the tube lens 118 optically coupled to the tunable filter 116, and the microscopy-adaptable camera 120 optically coupled to the tube lens 118.

In other words, the detection path 112 of the multimodal microscopy system 100 may be formed by the arrangement of the collection probe 122 (optional), the objective lens 114, the tunable filter 116, the tube lens 118, and the microscopy-adaptable camera 120. The light beam transmitted through or reflected by the sample (e.g. 106) may travel along the detection path (e.g. 112) to be collected and processed.

In various embodiments, the tunable filter 116 may be or may include a liquid crystal tunable filter or an acousto-optic tunable filter. Other filters that perform similar functions to the tunable filter 116 may be adopted in other embodiments. The tunable filter 116 may be free from having moving parts. Advantageously, the three hyperspectral imaging modalities described herein may share the same tunable filter 116 and may in turn share the same optical arrangement 110 and detection path (e.g. 112).

In one example, the tunable filter 116 may be or may include a band-pass filter configured to transmit a respective spectral band or a specific frequency of the light beam received from the objective lens 114.

In various embodiments, the microscopy-adaptable camera 120 may be a monochrome metal-oxide-semiconductor (CMOS) camera.

In various embodiments, the light guide 142 may be a liquid light guide.

FIG. 1(c) shows a schematic diagram illustrating the illumination module 102 of the multimodal microscopy system 100, according to some embodiments.

As seen in FIG. 1(c), the illumination module 102 may include a light source 140 for providing the light beam and may optionally include a light guide 142 optically coupled with the light source 140 for directing the light beam from the light source 140 to illuminate the sample (e.g. 106). For example, the light guide 142 may be employed when the multimodal microscopy system 100 is configured to perform brightfield hyperspectral microscopy or endoscopic hyperspectral microscopy. When performing fluorescent hyperspectral microscopy, a light guide may not be required.

### Multimodal Microscopy System Configured for Brightfield Hyperspectral Microscopy

For example, the illumination module 102 may include the light source 140 for providing the light beam; and the light guide 142 optically coupled with the light source 140 for directing the light beam from the light source 140 to illuminate the sample (e.g. 106), wherein the illumination module 102 may be configured to operate between a transmission mode and a reflection mode when the plurality of imaging modalities is switched to brightfield microscopy, for example, brightfield hyperspectral microscopy. In the transmission mode, the illumination module 102 may further include a condenser lens optically coupled with the light guide 142 to provide the light beam from underneath the sample (e.g. 106), and in the reflection mode, the illumination module 102 may further include collimation optics arranged in the delivery path between the light guide 142 and the objective lens 114 to deliver the light beam from above and onto the sample (e.g. 106). The light source 140 may be a broadband light source.

In more details, FIG. 2(a) shows a perspective view of a multimodal microscopy system 200 configured for brightfield hyperspectral microscopy set up in a transmission mode 144, in accordance with various embodiments. FIG. 2(b) shows a side partial view of the multimodal microscopy system 200 of FIG. 2(a) when in operation with a light path and in transmission mode.

The multimodal microscopy system 200, illumination module 202, optical arrangement 210, objective lens 214, tunable filter 216, tube lens 218, microscopy-adaptable camera 220, broadband light source 240, and light guide 242 in FIGS. 2(a) to 2(d) may be respectively or correspondingly described in a similar context to the multimodal microscopy system 100, illumination module 102, optical arrangement 110, objective lens 114, tunable filter 116, tube lens 118, microscopy-adaptable camera 120, light source 140, and light guide 142 of FIGS. 1(a) to 1(c).

As seen in FIG. 2(a), when operating in transmission mode 144 of the configured brightfield hyperspectral microscopy, the illumination module 202 may include the broadband light source 240 for providing the light beam, the light guide 242 optically coupled with the broadband light source 240 for directing the light beam from the broadband light source 240 to illuminate the sample 106, and a condenser lens 143 optically coupled with the light guide 242 to provide the light beam from underneath the sample 106. The light guide may be a liquid light guide, a single mode optical fiber, or a multi-mode optical fiber.

As shown in FIG. 2(b), when operating in transmission mode, light from the broadband light source 240 (not depicted in FIG. 2(b)) passes through the condenser lens 143, the sample 106, the objective lens 214, the tunable filter 216, the tube lens 218 and to a microscopy-adaptable camera 220.

FIG. 2(c) shows a perspective view of the multimodal microscopy system 200 configured for brightfield hyperspectral microscopy set up in a reflection mode 146, according to various embodiments. FIG. 2(d) shows a side partial view of the multimodal microscopy system 200 of FIG. 2(c) when in operation with a light path and in reflection mode.

In various embodiments, when operating in the reflection mode 146 of the brightfield hyperspectral microscopy, the illumination module 202 may include the broadband light source 240 for providing the light beam, the light guide 242 optically coupled with the broadband light source 240 for directing the light beam from the broadband light source 240 to illuminate the sample 106, and the collimation optics 145 arranged in the delivery path 108 between the light guide 242 and the objective lens 214 to deliver the light beam from above and onto the sample 106. In various embodiments, the collimation optics 145 may include a collimator 149 and a beam splitting member 147, wherein the collimator 149 is optically coupled with the beam splitting member 147. The beam splitting member 147 and the objective lens 214 are coupled to receive the light beam.

In the context of various embodiments, the collimation optics 145 may be configured to focus aligned light beams from the light guide 242 to a point and direct the focused light beams through the objective lens 214 to the sample 106. The beam splitting member 147 may be optically coupled to the objective lens 214.

As shown in FIG. 2(d), in various embodiments, when operating in the reflection mode 146, light from the light guide 242 (not shown in FIG. 2(d)) passes through the beam splitting member 147, the objective lens 214, the sample 106, back to the objective lens 214, through the beam splitting member 147 that directs the reflected light beams from the sample 106 through to the tunable filter 216, the tube lens 218 and to the microscopy-adaptable camera 220 along the detection path 112.

In various embodiments, the broadband light source 240 of the brightfield hyperspectral microscopy when operating in the transmission mode 144 or the reflection mode 146 may have a wavelength ranging from 420 nm to 1700 nm.

In various embodiments, the beam splitting member (e.g. 147) may include a dichroic mirror or a beam splitter.

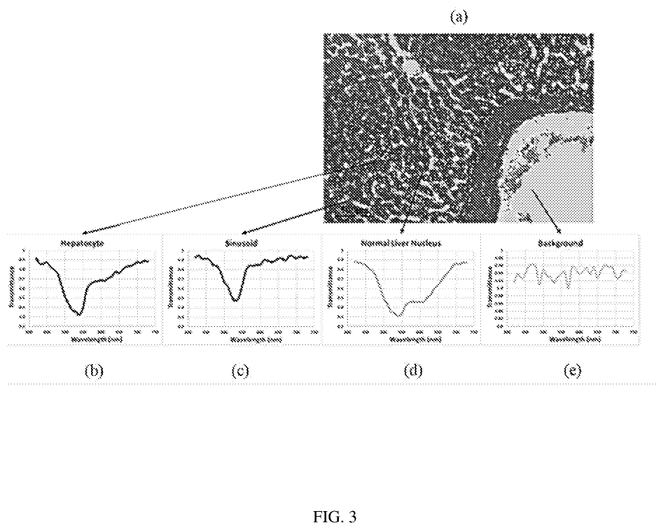

Some experiments have been carried out to compare the performance of the multimodal microscopy system 200 configured for brightfield hyperspectral microscopy and the results are described here. FIG. 3(a) shows a hyperspectral image of ex-vivo normal human liver cells with a false-color display where the colors represent material compositions. Each material composition is assumed to have a unique spectrum. FIGS. 3(b) to 3(e) are charts illustrating the spectral information of selected pixels in the image of FIG. 3(a).

FIG. 3(a) provides high-resolution spectral information of the normal human liver cells in the visible light wavelength range. As shown in FIGS. 3(b) to 3(e), according to the different transmission features, hepatocyte (FIG. 3(b)), sinusoid (FIG. 3(c)), normal liver nucleus (FIG. 3(d)), and background (FIG. 3(e)) information of normal human liver cells may be identified.

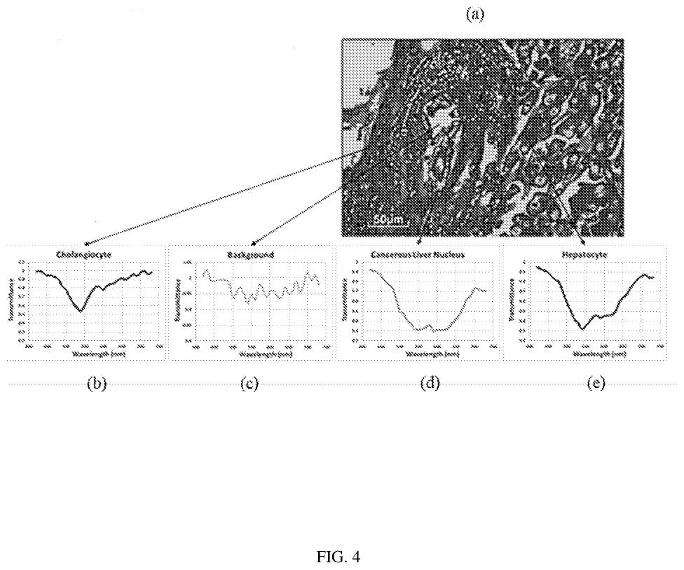

FIG. 4(a) shows a hyperspectral image of an ex-vivo cancerous human liver cell with a false-color display where the colors (shades) represent material compositions. Each material composition is assumed to have a unique spectrum.

FIGS. 4(b) to 4(e) are charts illustrating the spectral information of selected pixels in the image of FIG. 4(a).

FIG. 4(a) provides high-resolution spectral information of the cancerous human liver cells in the visible light wavelength range. As shown in FIGS. 4(b) to 4(e), according to the different transmission features, cholangiocyte (FIG. 4(b)), background (FIG. 4(c)), cancerous liver nucleus (FIG. 4(d)), and hepatocyte (FIG. 4(e)) information of cancerous human liver cells may be identified.

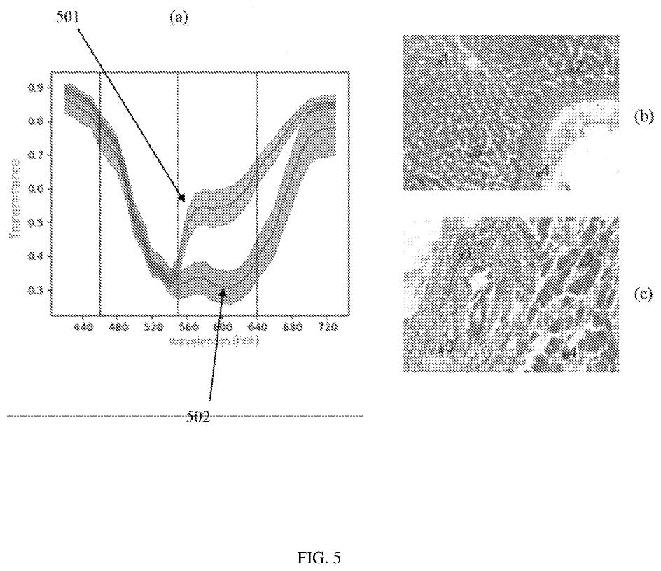

FIG. 5(a) is a graph illustrating the spectral reference library consisting of the transmittance spectrum of a normal liver and a cancerous liver. FIGS. 5(b) and 5(c) show true-color images of normal liver cells and cancerous liver cells, respectively, where the transmission features (i.e., spectral information) are used to distinguish normal and cancerous liver nuclei. Specifically, the solid lines in FIG. 5(a) are based on the averaged values of selected pixel points 1-4 showing nuclei in FIGS. 5(b) and 5(c). The shaded regions in FIG. 5(a) are the standard deviations.

As shown in FIG. 5(a), the transmission ratios of normal liver nuclei 501 and cancerous liver nuclei 502 may be distinguished within a visible light wavelength range.

### Multimodal Microscopy System Configured for Fluorescent Hyperspectral Microscopy

With reference to FIGS. 1(a) to 1(c), in various embodiments, when the plurality of imaging modalities may be switched to fluorescent microscopy, for example, fluorescent hyperspectral microscopy, the illumination module 102 may include the light source 140 for providing the light beam; and may be configured to operate between a transmission mode and a reflection mode. In the transmission mode 144, the illumination module 102 may further include a condenser lens optically coupled with the light source 140 to provide the light beam from underneath the sample (e.g. 106), and in the reflection mode, the illumination module may further include a filter cube arranged in the delivery path between the light source 140 and the objective lens 114 to deliver the light beam from above and onto the sample (e.g. 106). Here, the light source 140 may include a narrowband light source or a broadband light source with a filter. The broadband light source may have a wavelength ranging from 200 nm to 419 nm for ultraviolet (UV) range, from 420 nm to 730 nm for visible range, and 731 nm to 1700 nm for near and short-wave infrared range. In other examples, the light source 140 may also include a visible or infrared narrowband light source or the broadband light source with the filter. The filter may be an excitation filter.

In detail, FIG. 6(a) shows a perspective view of the multimodal microscopy system 200 configured for fluorescence hyperspectral microscopy set up in a transmission mode 144, according to various embodiments. FIG. 6(b) shows a side partial view of the multimodal microscopy system 200 of FIG. 6(a) when in operation with a light path.

As seen in FIG. 6(a), when the multimodal microscopy system 200 is switched to be operated in the transmission mode 144 of the fluorescent hyperspectral microscopy, the illumination module (e.g. 102 in FIG. 1(a)) may include a narrowband light source 240′ for providing the light beam and a condenser lens 143 optically coupled with the narrowband light source 240′ to provide the light beam from underneath the sample 106. As discussed above, instead of the narrowband light source 240′, the broadband light source with the filter (not shown in FIG. 6(a)) may be used.

As shown in FIG. 6(b), when operating in transmission mode 146, light from the narrowband light source 240′ (not shown in FIG. 6(b)) passes through the condenser lens 143, the sample 106, the objective lens 214, the tunable filter 216, the tub lens 218, and to the microscopy-adaptable camera 220.

FIG. 6(c) shows a perspective view of the multimodal microscopy system 200 configured for fluorescence hyperspectral microscopy set up in a reflection mode 146, according to various embodiments. FIG. 6(d) shows a side partial view of the multimodal microscopy system 200 of FIG. 6(c) when in operation with a light path. When operating in the reflection mode 146 of the fluorescent hyperspectral microscopy, the illumination module (e.g. 102 in FIG. 1(a)) may include the narrowband light source 240′ for providing the light beam that is directed to illuminate the sample 106, and a filter cube 245 being arranged in the delivery path 108 between the narrowband light source 240′ and the objective lens 214 to deliver the light beam from above and onto the sample 106. Instead of the narrowband light source 240′, the broadband light source with the filter (not shown in FIG. 6(d)) may be used.

As shown in FIG. 6(d), the filter cube 245 includes a collimator 149 configured to focus the light beam, an excitation filter 148 optically coupled to the collimator 149, and a beam splitting member 147 optically arranged between the tunable filter 216 and the collimator 149. The filter cube 245 may be regarded similarly to the collimation optics 145 of FIG. 2(d) with the addition of the filter 148 that may be an excitation filter. In a different example (not shown in the figures), when the narrowband light source 240′ is used, there may not be a need for the excitation filter 148, and thus, the collimation optics 145 of FIG. 2(d) instead of the filter cube 245 may be used. In yet another example, when the broadband light source with the filter is used, this filter may be the excitation filter 148 that may be disposed within the filter cube 245.

When operating in the reflection mode 146, light from the broadband light source 240 or the narrowband light source 240′ passes through the filter cube 245, the objective lens 214, the sample 106, back to the objective lens 214, through the beam splitting member 147 that directs the reflected light beams from the sample 106 through to the tunable filter 216, the tube lens 218 and to the microscopy-adaptable camera 220 along the detection path 112.

Here, the filter cube 245 may be arranged in the delivery path 108 between the broadband light source 240 or the narrowband light source 240′ and the objective lens 214 to deliver the light beam from above and onto the sample 106.

The beam splitting member 147 and the objective lens 214 are coupled to receive the light beam. The beam splitting member (e.g. 147) may include a dichroic mirror or a beam splitter.

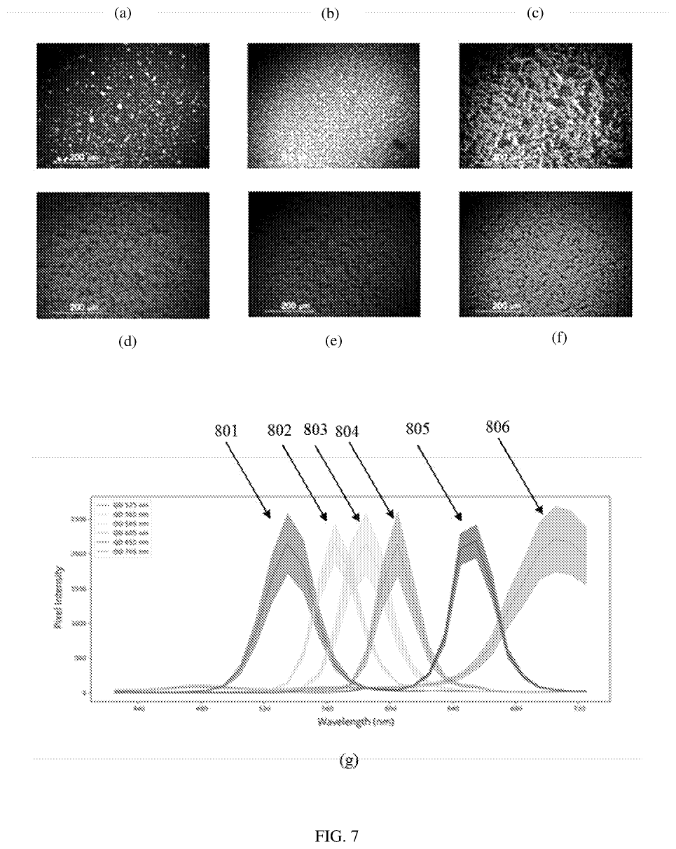

Some experiments have been carried out to assess the performance of the multimodal microscopy system 200 configured for fluorescent hyperspectral microscopy, and the results are described here. FIGS. 7(a) to 7(f) are true-color hyperspectral images, under 20× magnification, of six individual quantum dots, taken by the multimodal microscopy system 200 configured for fluorescent hyperspectral microscopy as described in FIG. 6(a). FIG. 7(g) shows emission spectral profiles of quantum dots of hyperspectral images of FIGS. 7(a) to 7(f).

As shown in FIG. 7(g), curves 801, 802, 803, 804, 805 and 806 are the spectral profiles of quantum dots with the peak emission wavelengths being 525 nm, 565 nm, 585 nm, 605 nm, 655 nm, and 705 nm, respectively.

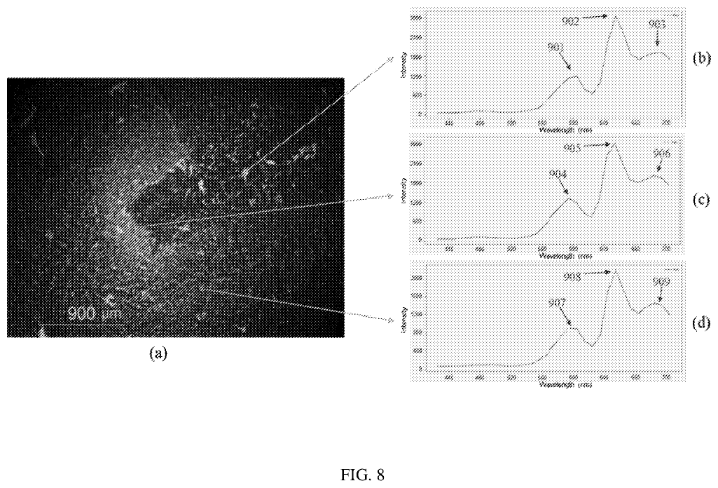

FIG. 8(a) is a false-color hyperspectral image, under 4× magnification, of the center of a mixture of six quantum dots labelled liver cells, taken by the multimodal microscopy system 200 configured for fluorescent hyperspectral microscopy in a transmission mode, as described in FIG. 6(a). FIGS. 8(b) to 8(d) are charts of spectra of selected pixels in the image of FIG. 8(a). The spectra are a combination of the spectrum of individual quantum dots based on the concentration or abundance of each type of quantum dot at that pixel. The concentration may vary at each pixel, so the spectra in FIGS. 8(b), 8(c), 8(d) are different. Peaks 901, 904, and 907 are mainly contributed by QD585 and QD605; peaks 902, 905, and 908 are mainly contributed by QD655; and peaks 903, 906, and 909 are mainly contributed by QD705.

### Multimodal Microscopy System Configured for Endoscopic Hyperspectral Microscopy

With reference to FIGS. 1(a) to 1(c), when the plurality of imaging modalities may be switched to endoscopic hyperspectral microscopy, the illumination module 102 may include the light source 140 for providing the light beam, the illumination module 102 being configured to operate between a transmission mode and a reflection mode and to provide external illumination. In the transmission mode, the illumination module 102 may further include a condenser lens (e.g. similarly described as 143 in FIG. 2(b)) and a light guide 142. The light guide 142 may be optically coupled with the light source 140 to provide the light beam from underneath the sample (e.g. 106). In the reflection mode, the illumination module 102 may further include a collimator (e.g. similarly described as 149 in FIG. 2(d)) and a light guide 142 optically coupled to the light source 140 placed at an angle to a collection probe to deliver the light beam above and onto the sample (e.g. 106). Here, the light source 140 may be a broadband or narrowband light source. With the broadband light source, brightfield measurements may be conducted under endoscopic modality. With the narrowband light source (or the broadband light source with the filter), fluorescent measurements may be conducted under endoscopic modality.

FIG. 9(a) shows a side view of a multimodal microscopy system configured for endoscopic hyperspectral microscopy in transmission mode with external illumination, according to one example. FIG. 9(b) shows a side view of a multimodal microscopy system configured for endoscopic hyperspectral microscopy in reflection mode with external illumination, according to another example.

As shown specifically in FIG. 9(a), in the transmission mode with external illumination, the illumination module (e.g. 102) may further include a collimator (e.g 149) and a light guide 742 optically coupled with the broadband or narrowband light source 740 to provide the light beam from underneath the sample 706 (which may be described in a similar context to 106 of FIGS. 2(a) to 2(d)). As shown specifically in FIG. 9(b), in the reflection mode with external illumination mode, the illumination module (e.g. 102) may further include a collimator (e.g. 149) and a light guide 742 optically coupled to the broadband or narrowband light source 740 placed at an angle to a collection probe 722 to deliver the light beam above and onto the sample 706. The broadband light source 740 may be described in a similar context to the broadband light source 240. The narrowband light source 740 source may be described in a similar context to the narrowband light source 240′.

The collection probe 722 may include a proximal end optically coupled to the objective lens 214 and a distal end, opposite to the proximal end. Through the distal end, the light beam from the sample 706 may be received via the detection path 112. The multimodal microscopy system configured for endoscopic hyperspectral microscopy may further include a three-axis stage configured to adjust a position of the proximal end of the collection probe 722 relative to that of the objective lens 214. For example, the three-axis stage may be provided by a z-axis stage 7024 and an x-y axis stage 7026.

The illumination module (e.g. 102) may alternatively be configured to operate in a reflection mode and to provide internal illumination when the plurality of imaging modalities is switched to endoscopic microscopy. Reference is made to FIGS. 10(a), 10(b) and 10(c) for better illustrations. FIG. 10(a) shows a side partial view 1000 of a fiber probe integrated with a metasurface 1102 at the facet thereof used for the multimodal microscopy system (e.g. 100) configured for endoscopic hyperspectral microscopy set up in a reflection mode with internal illumination. FIG. 10(b) shows a geometric representation of the metasurface 1102, while FIG. 10(c) shows a side view of the multimodal microscopy system with the fiber probe that is shown expanded in FIG. 10(a). Here, the illumination module (e.g. 102) may include a broadband or narrowband light source 740 (FIG. 10(c)) for providing the light beam, and the fiber probe including at least one optical fiber 1104 having a proximal end optically coupled to the broadband or narrowband light source (e.g. 740), and a distal end opposite to the proximal end, the distal end having the metasurface 1102 through which the light beam is to be directed above and onto the sample 706. The fiber probe may be rigid or flexible.

Taking specific reference to FIG. 10(b), the metasurface 1102 may be designed for off-axis focusing based on an equation of:

$$φ ( x , y , f , λ ) = \frac{2 π}{λ} n \times ( f - \sqrt{{( x - {x}_{f} )}^{2} + {( y - {y}_{f} )}^{2} + {z}_{{f}^{2}}} )$$

wherein x and y are spatial coordinates, f is a focal length, λ is a wavelength, x_{f}, y_{f}, and z_{f} are off-axis values where each of x_{f} and y_{f} may be a positive value or a negative value, n is the refractive index of an ambient medium.

As seen in FIG. 10(a), the fiber probe may further include a collection probe 1106 having a proximal end optically coupled to the objective lens (e.g. 214) and a distal end opposite to the proximal end. The collection probe 1106 may interchangeably refer to the collection probe 722 of FIG. 10(c) where through the distal end (shown within the side partial view 1000 of FIG. 10(a)), the light beam from the sample 706 may be received via the detection path 112 (e.g. similar to that as seen in FIG. 9(b)).

The distal end of the collection probe 1106 may be coupled to an optical component for focusing to facilitate non-contacting imaging and on-axis focusing. The optical component for focusing may be a gradient-index lens, or a ball lens, or may be provided by the metasurface 1102. The proximal end of the collection probe 1106 may be arranged along an imaging plane substantially same as that of the objective lens (e.g. 214). A three-axis stage (e.g. similar to those of FIG. 9(b)) may be provided and configured to adjust a position of the proximal end of the collection probe 1106 relative to that of the objective lens. For example, the three-axis stage may be provided by a z-axis stage 7024 and an x-y axis stage 7026.

It should be appreciated that for internal illumination, only reflection mode is applicable. Further, the metasurface 1102 may provide for the focal spots of the optical fiber 1104 and the collection probe 1106 to coincide on the sample 706, as shown in FIGS. 10(a) and 10(b).

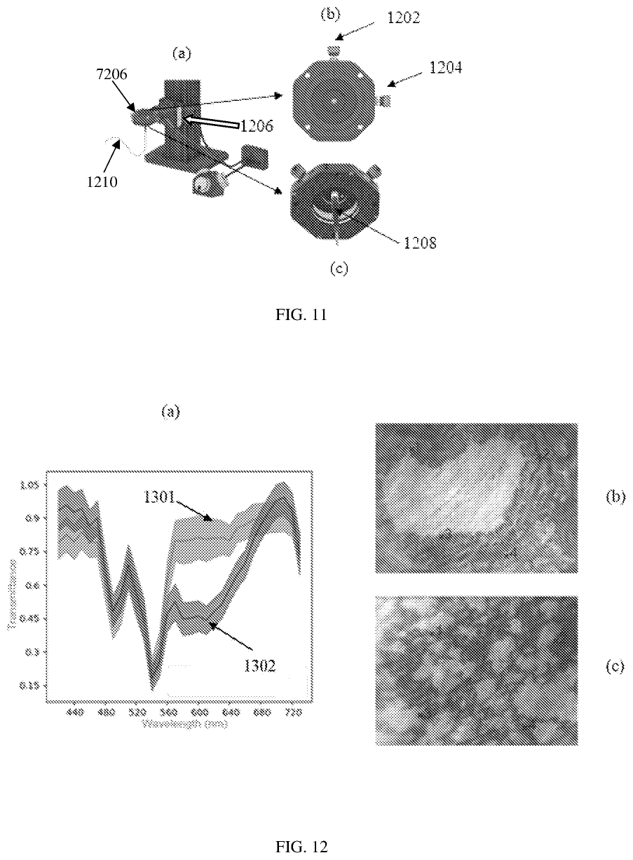

FIG. 11(a) shows a partial perspective view of a connection between the fiber probe (or more specifically, the collection probe 722, 1106) and a microscope body via the x-y axis stage 7206, according to one example. FIGS. 11(b) and 11(c) respectively show top and bottom perspective views of the connections between the fiber probe (or more specifically, the collection probe 722, 1106) and an imaging plane of the objective lens 214 (not shown in FIGS. 11(a) to 11(c)), as expanded from parts of FIG. 11(a). The x-y axis stage stage 7206 may be configured to adjust the positions along the x-axis, and y-axis as well as the z-axis (with 1206) to place the fiber probe 1210, that may be the collection probe 722 of FIGS. 9(a) and 9 (b) or the fiber probe including the collection probe 1106 of FIG. 10(a), at the imaging plane of the microscope objective lens (e.g. 214 in FIGS. 9(a) and 9(b)). The top view of the x-y axis stage 7206 may be seen in FIG. 11(b), in which a first adjustment knob 1202 may be arranged and used to control the movement of the x-y axis stage 7206 in the x-axis direction and a second adjustment knob 1204 may be arranged and used to control the movement of the x-y axis stage 7206 in the y-axis direction. A third adjustment knob 1206 may be arranged and used to control the movement of the x-y axis stage 7206 in the z-axis direction. A fiber connector 1208 may be provided to couple or connect the fiber probe 1210 with the x-y axis stage 7206.

Turning back to FIGS. 10(a) and 10(b), it may be seen that the optical fiber 1104 to illuminate the sample 706 may be placed side by side with the collection probe 1106, not externally at an angle (as seen in FIG. 9(b)). The collection probe 1106 may be an optical fiber bundle or a single fiber collecting reflected light from the sample 706 to the proximal end of the collection probe 1106. The reflected light may then be transmitted towards the distal end of the collection probe 1106 and to the objective lens 214. The metasurface 1102 on the fiber facet with off-axis focusing design enables the optical fiber 1104 provided for illuminating the sample 706 to be placed next or adjacent to the collection probe 1106 so that the footprint of the whole endoscope probe may be smaller as compared to the configuration providing external illumination (as seen in FIG. 9(b)) where the light guide is external and placed at an angle. As previously discussed, the metasurface may also allow focal spots of the optical fiber 1104 and collection probe 1106 to coincide on the sample 706, thereby enabling high resolution imaging and sensing.

In other words, the metasurface 1102 may be designed to provide off-axis focused illumination so that the optical fiber 1104 provided for illuminating the sample 706 may be placed substantially parallel close to the collection probe 1106. This may reduce the overall setup dimension, and this may be useful for applications where the size of the endoscope probe end may be critical.

For the multimodal microscopy system configured for endoscopic hyperspectral microscopy with external illumination (as in FIGS. 9(a) and 9(b)) or internal illumination (as in FIGS. 10(a), 10(b) and 10(c)), the distal end of the collection probe 722, 1106 may be added with a focusing optical component, such as a gradient-index (GRIN) lens so that the focused spots of the illumination and the collection coincide to provide high-resolution confocal imaging. The integration of endoscopic modality achieves high precision and high resolution imaging with a small footprint.

Similar to when the plurality of modalities is switched to brightfield hyperspectral microscopy, the broadband light source 740 of the endoscopic hyperspectral microscopy may have a wavelength ranging from 420 nm to 730 nm for visible range and 731 nm to 1700 nm for near and short-wave infrared range. Here, the collimation optics and the light guide 742 may be described in similar context of those used for brightfield hyperspectral microscopy, and thus descriptions thereon will be omitted here.

The light guide 742 may be a liquid light guide, a single mode optical fiber, or a multi-mode optical fiber.

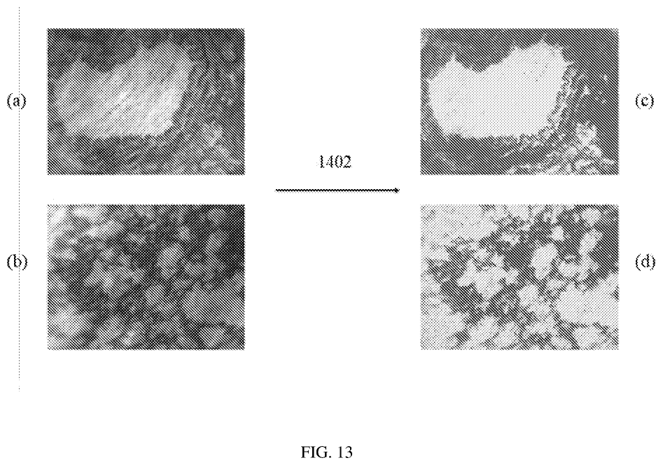

Some experiments have been carried out to assess the performance of the multimodal microscopy system configured for endoscopic hyperspectral microscopy, and the results are described here. FIG. 12(a) shows a graph illustrating the spectral reference library consisting of transmittance spectra of normal lung tissue 1301 and cancerous lung tissue 1302 averaged at selected points. FIGS. 12(b) and 12(c) show the reconstructed true-colour (RGB) images from the hyperspectral datacubes of normal lung tissue and cancerous lung tissue, respectively, taken by the multimodal microscopy system configured for endoscopic hyperspectral microscopy as described in FIG. 9(a), where each selected nuclei position is marked with a cross. For comparison purposes, FIG. 13(a) re-iterates the reconstructed true-colour (RGB) image of normal lung tissue of FIG. 12 (b), while FIG. 13(b) re-iterates the reconstructed true-colour (RGB) image of cancerous lung tissue of FIG. 12(c).

FIGS. 13(c) and 13(d) are the false-color representations of results of classified normal lung tissue and cancerous lung tissue, respectively by applying spectral analysis 1402 and using the reference library (FIG. 12(a)). Classification was calculated based on comparing the spectrum of each pixel to the reference library and the similar pixels are represented as one class (e.g. normal or cancerous) and shown as one colour. These results demonstrate the high resolution endoscopic imaging combined with hyperspectral imaging coupled with classification features.

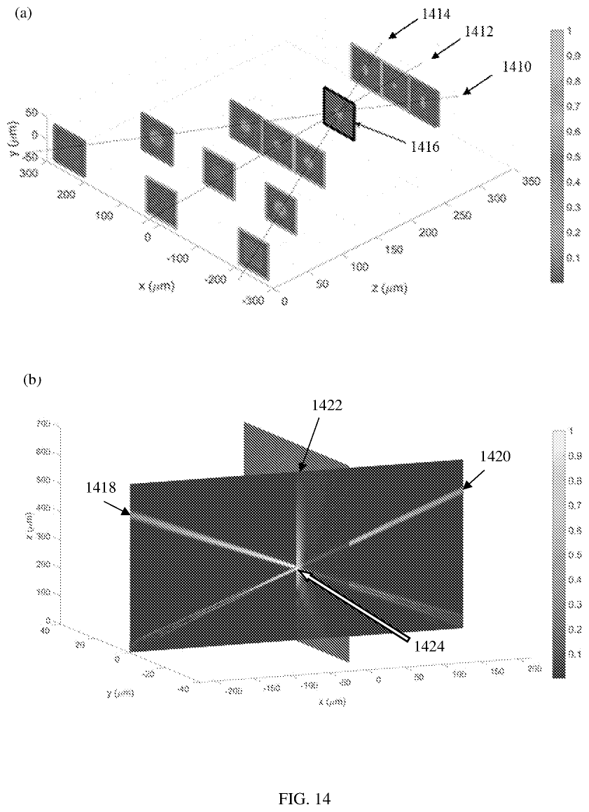

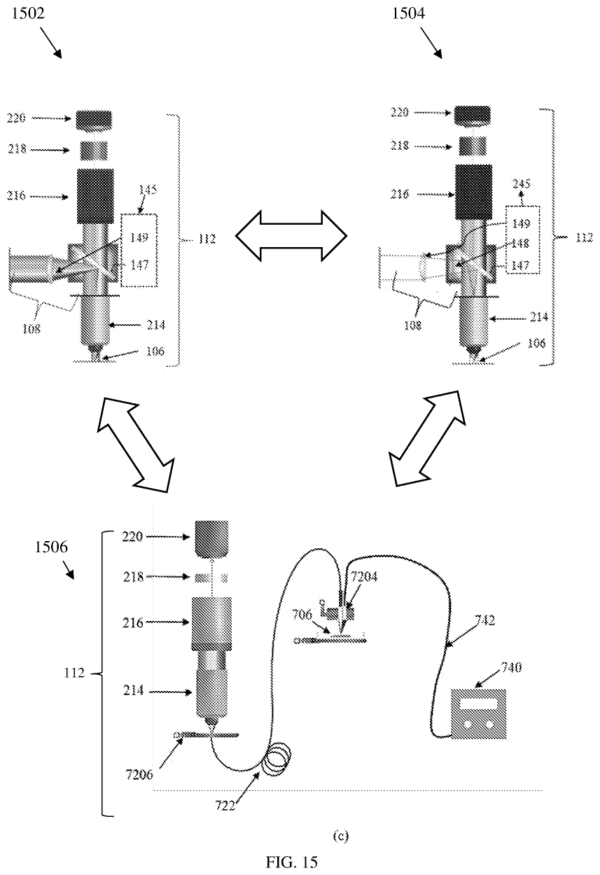

FIG. 14(a) shows a graphical representation of simulated 3D light beam propagations of two excitation beams (off-axis focusing) and one collection beam (on-axis focusing) modelled based on fiber points with metasurface integration, according to one example. As seen in FIG. 14(a), stimulated 3D light beam propagations of two excitation beams 1410, 1414 of off-axis focusing and one collection beam 1412 of on-axis focusing coincide on the same focal plane 1416 on the sample 706. The two excitation beams 1410, 1414 and one collection beam 1412 are modelled based on fiber points with metasurface integration. FIG. 14(b) shows a perspective view of the overlapping region of the stimulated three light beams, including excitation beams 1418, 1420 of off-axis focusing and one collection beam 1422 of on-axis focusing. The overlapping region is the focal point 1424. The excitation beams 1418, 1420 and the collection beam 1422 of FIG. 14(b) may be described in similar context to the two excitation beams 1410, 1414 and the collection beam 1412 of FIG. 14(a), respectively.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims and therefore intended to be embraced.

1. A multimodal microscopy system comprising:
an illumination module configured to provide a light beam to a sample under test via a delivery path; and
an optical arrangement configured to receive the light beam from the sample through a detection path to generate a microscopy image of the sample,
wherein the optical arrangement is operable to switch among a plurality of imaging modalities using the detection path shared by the plurality of imaging modalities, and
the plurality of imaging modalities comprises brightfield microscopy, fluorescent microscopy, and endoscopic microscopy.

2. The multimodal microscopy system as claimed in claim 1, wherein the optical arrangement comprises:
an objective lens configured to receive the light beam from the sample;
a tunable filter optically coupled to the objective lens, the tunable filter operable to select a wavelength or intensity or a range of wavelengths or intensities of the light beam;
a tube lens optically coupled to the tunable filter; and
a microscopy-adaptable camera optically coupled to the tube lens for capturing the microscopy image of the sample.

3. The multimodal microscopy system as claimed in claim 2, wherein the tunable filter is a liquid crystal tunable filter or an acousto-optic tunable filter, and the tunable filter is free from having moving parts.

4. The multimodal microscopy system as claimed in claim 2, wherein the illumination module comprises
a broadband light source for providing the light beam; and
a light guide optically coupled with the broadband light source for directing the light beam from the broadband light source to illuminate the sample, the illumination module configured to operate between a transmission mode and a reflection mode when the plurality of imaging modalities is switched to brightfield microscopy;
wherein in the transmission mode, the illumination module further comprises a condenser lens optically coupled with the light guide to provide the light beam from underneath the sample, and
in the reflection mode, the illumination module further comprises collimation optics arranged in the delivery path between the light guide and the objective lens to deliver the light beam from above and onto the sample.

5. The multimodal microscopy system as claimed in claim 4, wherein the collimation optics comprises:
a collimator configured to focus the light beam from the broadband light source; and
a beam splitting member optically coupled to an objective lens.

6. The multimodal microscopy system as claimed in claim 2,
wherein the illumination module comprises a broadband light source with a filter or a narrowband light source for providing the light beam; and is configured to operate between a transmission mode and a reflection mode when the plurality of imaging modalities is switched to fluorescent microscopy;
wherein in the transmission mode, the illumination module further comprises a condenser lens optically coupled with the broadband light source with the filter or the narrowband light source to provide the light beam from underneath the sample, and
in the reflection mode, the illumination module further comprises a filter cube arranged in the delivery path between the broadband light source with the filter or the narrowband light source and the objective lens to deliver the light beam from above and onto the sample.

7. (canceled)

8. The multimodal microscopy system as claimed in claim 6, wherein the filter cube comprises:
a collimator configured to focus the light beam;
an excitation filter optically coupled to the collimator; and
a beam splitting member optically arranged between the tunable filter and the collimator.

9. The multimodal microscopy system as claimed in claim 5, wherein the beam splitting member comprises a dichroic mirror or a beam splitter.

10. The multimodal microscopy system as claimed in claim 2,
wherein the illumination module comprises a broadband or narrowband light source for providing the light beam, the illumination module being configured to operate between a transmission mode and a reflection mode and to provide external illumination when the plurality of imaging modalities is switched to endoscopic microscopy;
wherein in the transmission mode, the illumination module further comprises a collimator and a light guide optically coupled with the broadband or narrowband light source to provide the light beam from underneath the sample, and
in the reflection mode, the illumination module further comprises a collimator and a light guide optically coupled to the broadband or narrowband light source placed at an angle to a collection probe to deliver the light beam above and onto the sample.

11. The multimodal microscopy system as claimed in claim 10, wherein the collection probe comprises a proximal end optically coupled to the objective lens, and a distal end opposite to the proximal end, the distal end through which the light beam from the sample is to be received via the detection path; and wherein the multimodal microscopy system further comprises a three-axis stage configured to adjust a position of the proximal end of the collection probe relative to that of the objective lens.

12. The multimodal microscopy system as claimed in claim 2,
wherein the illumination module comprises:
a broadband or narrowband light source for providing the light beam, the illumination module being configured to operate in a reflection mode and to provide internal illumination when the plurality of imaging modalities is switched to endoscopic microscopy; and
a fiber probe comprising at least one optical fiber having a proximal end optically coupled to the broadband or narrowband light source, and a distal end opposite to the proximal end, the distal end having a metasurface through which the light beam is to be directed above and onto the sample.

13. The multimodal microscopy system as claimed in claim 12, wherein the fiber probe further comprises a collection probe having a proximal end optically coupled to the objective lens, and a distal end opposite to the proximal end, the distal end through which the light beam from the sample is to be received via the detection path.

14. The multimodal microscopy system as claimed in claim 13, wherein the distal end of the collection probe is coupled to an optical component for focusing to facilitate non-contact imaging and on-axis focusing.

15. The multimodal microscopy system as claimed in claim 14, wherein the optical component for focusing is a gradient-index lens, or a ball lens, or is provided by the metasurface.

16. The multimodal microscopy system as claimed in claim 13, wherein the proximal end of the collection probe is arranged along an imaging plane substantially same as that of the objective lens.

17. The multimodal microscopy system as claimed in claim 13, further comprising a three-axis stage configured to adjust a position of the proximal end of the collection probe relative to that of the objective lens.

18. (canceled)

19. The multimodal microscopy system as claimed in claim 4, wherein the light guide is a liquid light guide, a single mode optical fiber, or a multi-mode optical fiber.

20. The multimodal microscopy system as claimed in claim 2, wherein the microscopy-adaptable camera is a monochrome complementary metal-oxide-semiconductor camera or a monochrome charged-coupled device camera.

21. (canceled)

22. The multimodal microscopy system as claimed in claim 8, wherein the beam splitting member comprises a dichroic mirror or a beam splitter.

23. The multimodal microscopy system as claimed in claim 10, wherein the light guide is a liquid light guide, a single mode optical fiber, or a multi-mode optical fiber.
